(19) United States
(12) Patent Application Publication
(10) Pub. No.: US 2025/0314650 A1
(43) Pub. Date: Oct. 9, 2025

(54) PHYSISORPTION OF ANTIBODIES ON FILMS

(71) Applicant: The General Hospital Corporation, Boston, MA (US)

(72) Inventors: Hyungsoon Im, Peabody, MA (US); Kihyeun Kim, Cambridge, MA (US)

(21) Appl. No.: 18/865,487

(22) PCT Filed: May 12, 2023
(86) PCT No.: PCT/US2023/022054

§ 371 Date: Nov. 13, 2024

Related U.S. Application Data
(60) Provisional application No. 63/341,838, filed on May 13, 2022.

(51) Int. Cl. G01N 33/543; G01N 33/531; G01N 33/545; G01N 33/552; G01N 33/553; G01N 33/574; G01N 33/68

(52) U.S. Cl. CPC G01N 33/54346; G01N 33/531; G01N 33/545; G01N 33/552; G01N 33/553; G01N 33/57484; G01N 33/6863; G01N 2333/52; G01N 2800/7028

(57) ABSTRACT

The present disclosure relates to direct immobilization of antibodies by physisorption onto plain and nanostructured metal-containing films. An exemplary method for preparing a sensor includes contacting an antibody with a surface of a film comprising an ionic compound and a metal selected from gold, silver, platinum, copper, and any combination thereof, and then contacting a blocking agent with the surface of the film to form the sensor.

### CLAIM OF PRIORITY

This application claims the benefit of U.S. Patent Application Ser. No. 63/341,838 filed on May 13, 2022. The entire contents of the foregoing are hereby incorporated by reference.

### FEDERALLY SPONSORED RESEARCH OR DEVELOPMENT

This invention was made with Government support under Grant Nos. CA217662 and GM138778 awarded by the National Institutes of Health. The Government has certain rights in the invention.

### TECHNICAL FIELD

Described herein are methods of physisorbing antibodies onto films, and sensors including antibodies and blocking agents physisorbed on a film.

### BACKGROUND

Extracellular vesicles (EVs) are membrane-bound nano-sized vesicles actively shed by cells into the circulation. EVs are abundantly present in biofluids, structurally stable, and carry biomolecules from their originating cells. These properties bring attention to EVs as attractive circulating biomarkers for the diagnosis of cancers, as well as neurodegenerative, cardiovascular, inflammatory, and infectious diseases.

### SUMMARY

Provided herein is a method of preparing a sensor, the method including contacting an antibody with a surface of a film including an ionic compound and a metal selected from gold, silver, platinum, copper, and any combination thereof, and then contacting a blocking agent with the surface of the film to form the sensor.

Also provided herein is a method of physisorbing an antibody onto a film, the method including contacting the antibody with a surface of the film consisting essentially of an ionic compound and a metal selected from gold, silver, platinum, copper, and any combination thereof.

Also provided herein is a sensor. The sensor includes a film having a sensing surface, an antibody physisorbed on the sensing surface, and a blocking agent physisorbed on the sensing surface. The film includes a metal selected from gold, silver, platinum, copper, and any combination thereof, and the metal makes up at least a portion of the sensing surface.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of physisorption and chemisorption procedures for antibody immobilization on gold substrates.

FIG. 2 is a set of fluorescence images of fluorescently labeled extracellular vesicles (EVs) captured on plain gold substrates coated with anti-CD63 and IgG control antibodies using physisorption and chemisorption methods. Scale bars=50 μm.

FIG. 3 is a set of graphs showing (left) the number of captured EVs on plain gold substrates prepared by different methods, and (right) specific capture efficiencies, defined by the ratio between specifically and nonspecifically captured EVs, for substrates coated using physisorption and chemisorption methods.

FIG. 4 is graph showing the number of nonspecifically bound EVs on differently immobilized antibody/gold film: physisorption and chemisorption using 11-mercaptoundecanoic acid (MUA) and polyethylene glycol (PEG).

FIG. 5 is a schematic illustration of interactions between antibodies and gold film via physisorption.

FIG. 6 is a schematic illustration of interactions between antibodies and linker/gold film in a chemisorption method.

FIG. 7 is a set of fluorescence images after immobilization of AF647 dye-labeled antibodies on gold substrates that were prepared by physisorption and chemisorption methods, showing the uniformity of antibody coating on the substrates. Scale bars=50 μm.

FIG. 8 is a scanning electron micrograph of a 100-nm thick gold film deposited by e-beam deposition on a glass substrate. Scale bar=200 nm.

FIG. 9 is a schematic illustration of interactions between (left) antibodies and a gold film and (right) antibodies and linkers defectively formed on the gold film.

FIG. 10 is a scanning electron micrograph of a gold nanowell substrate.

FIG. 11 is a set of set of fluorescence images showing specifically bound OV90 EVs on anti CD63 antibody-and IgG control antibody-immobilized nanowell substrates, respectively, which were prepared by physisorption and chemisorption methods (scale bars=25 μm).

FIG. 12 is a set of fluorescence images showing non-specifically bound OV90 EVs on anti CD63 antibody-and IgG control antibody-immobilized nanowell substrates, which were prepared by physisorption and chemisorption methods (scale bars=25 μm).

FIG. 13 is a set of graphs showing the number of nonspecifically bound EVs on isotope IgG antibody-coated nanowell substrates that were prepared using (left) physical and (center) MUA-based and (right) PEG-based chemical methods (n=6).

FIG. 14 is a graph showing the numbers of captured EVs with titrating concentrations of OV90 EVs on gold nanowell substrates coated with anti-CD63 antibodies.

FIG. 15 is a graph showing the numbers of captured EVs with titrating concentrations of OV90 EVs on gold nanowell substrates coated with IgG control antibodies.

FIG. 16 is a graph showing net EV counts after subtracting the numbers of nonspecifically bound EVs from those from anti-CD63 coated substrates.

FIG. 17 is a set of fluorescence images of nanowell substrates functionalized with anti-CD63, anti-EpCAM, and IgG control antibodies. EVs from OV90 ovarian cancer cells were fluorescently labeled by AZDye 555 (shown green in the images). EVs from TIOSE4 benign cells were fluorescently labeled by AZDye 647 (shown red in the images). Scale bars=50 μm.

FIG. 18 is a graph showing captured EV counts for a mixture of OV90 and TIOSE4-derived EVs were applied on anti-CD63, anti-EPCAM, and IgG coated nanowell substrates.

FIG. 19 is a graph showing specific capture efficiencies, defined by the ratio between specifically and nonspecifically captured EVs, for OV90 and TIOSE4-derived EVs.

FIG. 20 is a graph showing the number of EVs from OV90 and CaOV3 ovarian cancer cells as well as healthy human plasma samples captured on anti-CD63, anti-EpCAM, and IgG antibody-coated nanowell substrates.

FIG. 21 is a graph showing specific capture efficiencies for EVs from OV90 and CaOV3 ovarian cancer cells as well as healthy human plasma samples.

FIG. 22 is a graph showing EV binding on anti-CD63 antibody-coated surfaces prepared by physisorption and chemisorption methods with MUA or PEG. Corresponding sets shown in FIGS. 22 and 23 proceeded at the same time.

FIG. 23 is a graph showing EV binding on IgG antibody-coated surfaces prepared by physisorption and chemisorption methods with MUA or PEG. Corresponding sets shown in FIGS. 22 and 23 proceeded at the same time.

FIG. 24 is a graph showing the number of OV90 EVs non-specifically bound on Ig-G coated gold substrates.

FIG. 25 is a set of graphs showing the number of cancer-derived EVs (OV90) captured on anti-CD63 or IgG antibody-coated gold substrates prepared by physisorption and chemisorption methods with MUA and PEG linkers.

FIG. 26 is a set of images showing the results of EV detection and counting: (left) fluorescence images in full size and zoomed in; (right) the results of EV detection showing yellow circles indicating the location of detected EVs. A total of 30,160 dots were detected in the full-size image.

FIG. 27 is a set of fluorescence images showing bound protein on anti-MCP-1 antibody-immobilized substrates prepared by physisorption (citrate-based) methods.

FIG. 28 is a graph showing the fluorescence intensity of anti-MCP-1 antibody-immobilized substrates prepared by physisorption (citrate-based) methods after contacting solutions including 0-10 pg/mL MCP-1.

FIG. 29 is a graph showing the fluorescence intensity of anti-MCP-1 antibody-immobilized substrates prepared by physisorption (citrate-based) or chemisorption (MUA-based) methods after contacting solutions including 0 or 1 ng/mL MCP-1.

FIG. 30 is a graph showing the number of tumor-derived EVs and HER2-positive tumor-derived EVs captured on QUAD capture antibody-coated gold substrates prepared by physisorption (citrate-based) methods.

FIG. 31 is a graph showing the number of tumor-derived EVs and HER2-positive tumor-derived EVs captured on QUAD capture antibody-coated gold substrates prepared by chemisorption methods with PEG linkers.

### DETAILED DESCRIPTION

Extracellular vesicle (EV) detection platforms include plasmonic sensors, which harness surface plasmon resonance (SPR) excited on metal substrates or nanostructures. The plasmonic sensing approach is simple, sensitive, and amenable to high throughput assays. Plasmonic sensors detect EVs by a resonance shift induced by the increase of local refractive index upon EV binding to a gold sensing surface, which allows for simple, rapid label-free detection. Furthermore, plasmonic sensors ‘sensing range (typically 10-300 nm) matches well with the size of the majority of EVs, boosting the sensitivity for EV detection. However, non-specific binding of non-target molecules to the plasmonic sensing surface can preclude reliable and robust EV detection. Such non-specific binding can also increase the local refractive index, causing false-positive signals and adversely affecting the sensor's limit of detection.

Gold, a popular material for plasmonic sensors, is vulnerable to non-specific molecular adsorption by hydrophobic and electrostatic interactions. Thus, self-assembly of thiol-based linkers (e.g., short carbon chains, polyethylene glycol (PEG), dextran polymers) with carboxyl functional groups are typically used to immobilize affinity ligands on gold films through a 1-ethyl-3-(3-dimethylaminopropyl) carbodiimide/N-hydroxysuccinimide (EDC/NHS) coupling reaction while passivating the surface to reduce non-specific bindings. Even with thiol-based linkers and passivation layers, non-specific binding can still occur because of (i) defective formation of linkers, and/or (ii) byproducts formation in the EDC/NHS reaction. The former can occur at defective sites (e.g., grain boundaries, step edges, and vacancies) on a gold surface and result in weak antibody attachment and surface passivation. Regarding the latter, byproducts, such as hydrolyzed carboxyl and N-acylurea groups from an EDC/NHS reaction, often interfere with the covalent binding of antibodies to the linkers. Such areas with defective linker layers and byproduct formation can be vulnerable for EVs ‘non-specific binding despite surface blocking with bovine serum albumin (BSA), fetal bovine serum (FBS), and other blocking reagents.

The present disclosure relates to direct immobilization of antibodies by physisorption onto plain and nanostructured metal-containing films. Sensors including physisorbed antibodies can provide reproducible, specific EV capture with reduced non-specific binding. For example, sensors formed by physisorption of antibodies on citrate-treated plain and nanowell gold films can demonstrate non-specific EV binding that is reduced by >50-fold compared to sensors formed by conventional chemisorption methods involving MUA or SH-PEG-COOH linkers. This low non-specific binding and good reproducibility can be attributed, at least in part, to the uniform coverage of the surface with physisorbed antibodies and blocking agents, and to methods that minimize variations in the physisorption process. The methods described herein can accordingly provide sensors that are more robust and more efficient for EV detection, as compared to conventional sensors including chemisorbed antibodies.

### Sensors

Provided herein are sensors that include a film having a sensing surface, an antibody physisorbed on the sensing surface, and a blocking agent physisorbed on the sensing surface. The sensing surface can include an ionic compound, e.g., a carboxylate-containing compound. Unlike chemisorption, which involves discrete covalent linkers, the physisorption described herein involves hydrophobic and electrostatic interactions directly between a metal-containing surface and the antibody or blocking agent. For example, direct hydrophobic and electrostatic interactions between an antibody and metal (e.g., gold) and/or an ionic compound (e.g., citrate) present at the sensing surface can contribute to physisorption of the antibody on the sensing surface.

The film includes a metal selected from gold, silver, platinum, copper, and any combination thereof, which metal makes up at least a portion of the sensing surface. In some embodiments, the metal makes up at least 70%, e.g., at least 80%, at least 90%, at least 95%, at least 97.5%, or at least 99% of the sensing surface of the film. In some embodiments, the metal is gold or silver. For example, in some embodiments, the metal is gold.

In some embodiments, the film includes an ionic compound, which ionic compound makes up at least a portion of the sensing surface. In some embodiments, the ionic compound includes an ammonium group, a carboxylate group, or an azide group. For example, in some embodiments, the ionic compound includes a citrate ion, an acetate ion, a succinate ion, a glutarate ion, or any combination thereof. In some embodiments, the ionic compound includes a citrate ion. For example, in some embodiments, the ionic compound is sodium citrate or potassium citrate. In some embodiments, the sensing surface consists essentially of the metal and the ionic compound. For example, in some embodiments, the sensing surface consists essentially of gold and a citrate.

In some embodiments, the antibody has a dissociation constant for the sensing surface of 0.1 nM to 500 nM, e.g., 0.1 nM to 150 nM, 0.1 nM to 75 nM, 1 nM to 500 nM, 1 nM to 150 nM, 1 nM to 75 nM, 5 nM to 500 nM, 5 nM to 150 nM, or 5 nM to 75 nM. In some embodiments, the antibody has a binding affinity for the sensing surface that is greater than a binding affinity of an extracellular vesicle for the sensing surface.

In some embodiments, the antibody is capable of binding a biomarker that is a cellular or molecular target relevant to, for example, cancers, neurodegenerative diseases, blood/vascular diseases, infection, inflammation, or wound healing. In some embodiments, the antibody is capable of binding to a cancer biomarker. In some embodiments, the antibody is capable of binding to a biomarker selected from EpCAM, EGFR, MUC-1, HER2, CD24, CA125, CD45, CD63, CD81, CD9, or DAPI. In some embodiments, the biomarker is present in an extracellular vesicle (e.g., on the surface of an extracellular vesicle). In some embodiments, the biomarker includes a cytokine, e.g., monocyte chemoattractant protein (MCP-1), IL-1B, IL-3, IL-10, TNFα, or interferon-γ. In some embodiments, the biomarker is a soluble protein (e.g., present in solution).

In some embodiments, the blocking agent has a dissociation constant for the sensing surface of 0.1 nM to 500 nM, e.g., 0.1 nM to 150 nM, 0.1 nM to 75 nM, 1 nM to 500 nM, 1 nM to 150 nM, 1 nM to 75 nM, 5 nM to 500 nM, 5 nM to 150 nM, or 5 nM to 75 nM. In some embodiments, the blocking agent has a binding affinity for the sensing surface that is greater than a binding affinity of an extracellular vesicle for the sensing surface.

In some embodiments, the blocking agent includes bovine serum albumin, fetal bovine serum, goat serum, steelhead salmon serum, non-fat milk, SuperBlock™ (Thermo Scientific), AdvanBlock™ (Advantsta Inc.), or any combination thereof. In some embodiments, the blocking agent is bovine serum albumin.

In some embodiments, any binding-accessible domains of the sensing surface (e.g., unoccupied by physisorbed antibody or by physisorbed blocking agent) have an average size of less than that necessary for an extracellular vesicle to bind to the sensing surface. In some embodiments, the average size of binding-accessible domains of the sensing surface is 1 nm to 50 nm, e.g., 1 nm to 25 nm, 1 nm to 10 nm, or 1 nm to 5 nm. In some embodiments, the binding-accessible domains of the sensing surface include the ionic compound.

In some embodiments, the sensing surface is flat. Such sensing surfaces include, for example, the surface of a metal film (e.g., a film including gold and a carboxylate-containing compound). In some embodiments, the sensing surface includes nano-scale features. Such sensing surfaces include, for example, the surface of a metal film (e.g., a film including gold and a carboxylate-containing compound) disposed over a nano-textured substrate (e.g., an etched silicon-containing substrate, such as a silicon nitride nanowell substrate). Such sensing surfaces also include, for example, the surface of an etched metal film (e.g., a nanopillared gold film).

In some embodiments, the nano-scale features include holes, ridges, channels, wells, pillars, pyramids, cones, particles, or any combination thereof. In some embodiments, the nano-scale features include wells (nanowells). In some embodiments, the wells have an average diameter of 50 nm to 1000 nm, e.g., 100 nm to 300 nm. In some embodiments, the wells have an average depth of 50 nm to 1000 nm, e.g., 100 nm to 300 nm. In some embodiments, the nano-scale features include pillars (nanopillars). In some embodiments, the pillars have an average diameter of 20 nm to 1000 nm, e.g., 100 nm to 300 nm. In some embodiments, the pillars have an average height of 20 nm to 1000 nm, e.g., 100 nm to 300 nm. In some embodiments, the nano-scale features include particles (nanoparticles). In some embodiments, the particles have an average diameter of 20 nm to 500 nm, e.g., 30 nm to 300 nm, or 50 nm to 200 nm. In some embodiments, the nano-scale features include particles and pillars. For example, in some embodiments, the nano-scale features include nanoparticles-on-nanopillars (NPOP).

In some embodiments, the nano-scale features make up a pattern (e.g., having one or more periodicities) on the sensing surface. For example, in some embodiments, adjacent nano-scale features (e.g., wells or pillars) are spaced apart by an average distance of 50 nm to 1 μm, e.g., 50 nm to 750 nm, 50 nm to 500 nm, 100 nm to 1 μm, 100 nm to 750 nm, or 100 nm to 500 nm.

In some embodiments, the film has an average thickness (e.g., inclusive of nano-scale features) of 10 nm to 1 μm, e.g., 10 nm to 750 nm, 10 nm to 500 nm, 50 nm to 1 um, 50 nm to 750 nm, 50 nm to 500 nm, 100 nm to 1 μm, 100 nm to 750 nm, or 100 nm to 500 nm. In some embodiments, the sensor includes a substrate, and the film is disposed on the substrate such that the sensing surface faces away from the substrate. In some embodiments, the substrate includes silicon, e.g., crystalline silicon or crystallize silicon nitride. In some embodiments, the substrate includes an adhesion layer, and the film is disposed on the adhesion layer. In some embodiments, the adhesion layer has an average thickness of 1 nm to 50 nm, e.g., 1 nm to 25 nm, or 1 nm to 10 nm. In some embodiments, the adhesion layer includes titanium. In some embodiments, the substrate includes glass or plastic.

In some embodiments, the sensor is a plasmon resonance sensor. In some embodiments, the sensor is an electrochemical sensor.

### Preparation Methods

Also provided herein are methods for physisorbing an antibody onto a film containing an ionic compound described herein and a metal selected from gold, silver, platinum, copper, and any combination thereof. In some embodiments of the methods, the metal is silver or gold. For example, in some embodiments of the methods, the metal is gold. The method can include contacting the antibody with a surface of the film consisting essentially of the metal and an ionic compound. In some embodiments, the film consists essentially of a layer of the ionic compound disposed on a layer of the metal.

Also provided herein is a method for preparing a sensor, such as a sensor described herein. The method includes contacting an antibody with a surface of a film including an ionic compound described herein and a metal selected from gold, silver, platinum, copper, and any combination thereof, and then contacting a blocking agent with the surface of the film to form the sensor.

In some embodiments of the methods, the metal makes up at least 70%, e.g., at least 80%, at least 90%, at least 95%, at least 97.5%, or at least 99% of the surface of the film. In some embodiments of the methods, the metal is gold or silver. For example, in some embodiments of the methods, the metal is gold.

In some embodiments, the method further includes, before contacting the antibody, forming the surface by contacting a precursor film comprising the metal with the ionic compound. In some embodiments of the methods, the precursor film consists essentially of the metal. In some embodiments, of the methods, forming the surface comprises contacting the precursor film with an aqueous solution of the ionic compound.

In some embodiments of the methods, the ionic compound includes an ammonium group, a carboxylate group, an azide group, or any combination thereof. For example, in some embodiments of the methods, the ionic compound includes citric acid, acetic acid, succinic acid, glutaric acid, any salt thereof, or any combination thereof. In some embodiments of the methods, the ionic compound is a salt of citric acid (e.g., sodium citrate or potassium citrate).

In some embodiments, the surface of the film consists essentially of the metal and an ionic compound. In certain such embodiments, the film consists essentially of a layer of the ionic compound disposed on a layer of the metal. Such surfaces can be formed, for example, by contacting the surface of a metal film (e.g., a gold film) with an aqueous solution of the ionic molecule (e.g., a citrate).

In some embodiments of the method, the surface of the film is flat. In some embodiments of the method, the surface of the film includes nano-scale features (e.g., as described herein).

In some embodiments of the method, the film is disposed on a substrate such that the contacting surface of the film faces away from the substrate. In some embodiments, the substrate is a substrate described herein.

In some embodiments, the method includes contacting the surface of the film with an aqueous solution of the antibody, and then contacting the surface of the film with an aqueous solution of the blocking agent.

### EXAMPLES

The invention is further described in the following examples, which do not limit the scope of the invention described in the claims.

### General Experimental Details

Materials: Tri-Sodium citrate dihydrate (United States Pharmacopeial/USP standards), sodium bicarbonate (ACS reagent, ≥99.7%), Phosphate buffer solution (0.1 M), Bovine serum albumin (heat shock fraction, protease free, fatty acid free, essentially globulin free, pH 7, ≥98%), RPMI 1640 Complete Medium, Sepharose® CL-4B, and 11-mercaptoundecanoic acid (MUA) were obtained from Sigma Aldrich (St. Louis, MO, USA). Alexa Fluor® 647 Mouse IgG1, K Isotype Ctrl (FC) was purchased from BioLegend (San Diego, CA, USA). Anti-CD63 antibody was obtained from Ancell Corporation (Minnesota, USA). Thiol PEG acid (SH-PEG-COOH, 1,000 Da) and thiol functionalized methoxyl polyethylene glycol, PEG thiol (mPEG-SH, 350 Da) were purchased from Nanocs Inc. (New York, USA). Phosphate buffered saline (PBS, pH 7.4), Tris buffer (1 M, pH 8.0, RNase-free), MES buffer (BupH™ MES Buffered Saline Packs), EpCAM Monoclonal Antibody (323/A3), Mouse IgG1 kappa Isotype Control (P3.6.2.8.1) antibody, Zeba™M Micro Spin Desalting Columns (40K MWCO, 75 μL), 1-ethyl-3-(3-dimethyl aminopropyl) carbodiimide hydrochloride (EDC), N-hydroxysulfosuccinimide (sulfo-NHS), dimethyl sulfoxide (anhydrous, 99.8+%) (DMSO), ethanol (99.5%, ACS reagent, absolute), Fetal Bovine Serum (exosome-depleted) (FBS), and ProLong™ Gold Antifade Mountant were purchased from Thermo Fisher Scientific (Waltham, MA, USA). Sterile human plasma in sodium EDTA (number: D519-04-0050) was obtained from Rockland (Pottstown, PA, USA). Nanodimple (200-nm hole size and 500-nm periodicity) Si3N4 substrate was purchased from LumArray, Inc. (Massachusetts, USA). AZDye 555 DBCO and AZDye 647 DBCO were obtained from Click Chemistry Tools (Arizona, USA). Azido-dPEGR_{1 2}-TFP ester was purchased from Quanta Biodesign (Ohio, USA).

Preparation of Gold Substrates: 100-nm-thick gold films with 5-nm titanium as an adhesion layer between the gold and the substrate were deposited using an e-beam evaporator on a silicon wafer and a nanowell patterned Si₃N₄ substrate to obtain plain and nanowell gold substrates, respectively.

Physisorption of Antibodies on Gold Substrates: The gold substrate was cleaned with ethanol and deionized water. Then the substrate was immersed in a 100 mM citrate solution for 30 min. An antibody solution (20 μg mL⁻¹ in 10 mM PB, pH 7.0) was pipetted onto the cleaned gold substrate and incubated for 1 h. Then, the surface of the substrate was blocked with a BSA solution (final concentration: 10 mg mL⁻¹ in 1×PBS) and incubated for 30 min followed by washing. Finally, the substrate was washed using 1×PBS.

Chemisorption of Antibodies on Gold Substrates: Two different linkers (MUA or SH-1k-PEG-COOH) were used for the chemisorption of antibodies on gold substrates. For MUA, the gold substrate was cleaned with ethanol, and then, the substrate was immersed in 10 mM MUA solution (in ethanol) for 12 h. It should be noted that the MUA solution was sonicated to disperse aggregated MUA in ethanol before it was used. After the formation of the MUA layer on the gold surface, the substrate was washed with ethanol under sonication. For SH-PEG-COOH, 1 mM SH-PEG-COOH solution (in pH 3.0 Tris buffer), 0.5 mM mPEG-SH (in pH 3.0 Tris buffer), and 0.1% SDS were mixed with the ratio of 1:1:1 and dropped onto the cleaned gold substrate. After incubation for 12 h, the PEG acid-coated gold substrate was washed using deionized water. After linker formation, a mixture solution containing EDC and NHS (final concentrations of EDC and NHS were 0.5 M in pH 6.0 MES buffer) was freshly prepared and dropped to MUA-coated gold substrate. After incubation for 10 min, the substrate was washed with deionized water. Then, 20 μg mL⁻¹ of antibody solution (diluted in pH 7.0 PB) was pipetted onto the substrate, and the substrate was incubated for 30 min. For blocking, a blocking solution (10 mg mL⁻¹ BSA and 0.5 M glycine in PBS) was added to the substrate and incubated for 30 min followed by washing. Then, the substrate was washed using PBS.

Cell Culture and Extracellular Vesicle (EV) Isolation: The human ovarian carcinoma cell lines (OV90 and CaOV3) were purchased from American Type Culture Collection (ATCC), and the ovarian benign cell line (TIOSE4) was obtained from transfection of hTERT into NOSE cells maintained in 1:1 Media 199:MCDB 105 with gentamicin (25 μg mL-1), 15% heat-inactivated serum, and G418 (500 μg mL-1). All cell lines were maintained in RPMI-1640 (Hyclone) complete medium supplemented with 10% FBS, 100 U mL-1 penicillin, and 100 μg mL-1 streptomycin (Cellgro) at 37°° C. in 5% CO2. EV isolation was conducted according to literature procedure. Briefly, the cells were cultured until 80-90% in a conditioned medium supplemented with 1% Exosome-depleted FBS, 100 U mL-1 penicillin, and 100 μg mL-1 streptomycin for 48 h. Next, the supernatant was collected and concentrated with Centricon Plus-70 Centrifugal Filter (MWCO=10 kDa). The concentrate was loaded onto a size-exclusion chromatography column which was packed with Sepharose CL-4B. The fractions of 4 and 5 of 1 mL were collected and concentrated with the Amicon Ultra-2 Centrifugal Filter (MWCO=10 kDa). The isolated EVs were stored until use at −80° C.

Plasma EV Isolation: Healthy normal plasma samples were obtained from MGH Biobank (IRB #2019P003472). The plasma samples were centrifuged at 300 g for 10 min at 4° C. to eliminate the dead cells and debris. After that, they were centrifuged at 2,000 g for 20 min at 4° C. again to separate the apoptotic bodies. The supernatant was passed through with size-exclusion chromatography for taking the 4th and 5th fractions. To concentrate the EV solution, Amino Ultra-2 Centrifugal Filter was used and centrifuged at 3,500×g for 30 min at 4° C.

Nanoparticle Tracking Analysis (NTA) for EV Concentration Measurements: The concentrations of isolated EVs were measured using a NanoSight LM10 microscope (Malvern). The measurements were conducted with a 642 nm laser module at room temperature. Each sample was diluted 500 fold in PBS and manually placed in the chamber. Briefly, each sample was recorded with a camera for 30 seconds in a quadruplicate and analyzed with NTA software.

Fluorescent Labeling of EVs: Isolated EVs were labeled with AZDye 555-PEG-TFP ester and AZDye 647-PEG-TFP ester. First, AZDye-PEG-TFP ester was prepared by mixing 5 μL of 25 mM AZDye 555 (or 647) DBCO in anhydrous DMSO with 5 μL of 27.5 mM Azido-dPEG also in anhydrous DMSO). The mixture was incubated at room temperature for 2 h. For EV labeling, 3 μL of EV solution (1.3×109 EVs mL-1) was mixed with 2 μL of 0.3 M sodium bicarbonate and 0.2 μL AZDye-PEG-TFP ester (10 mg mL-1 in DMSO) and incubated for 1 h. The fluorophore-labeled EVs were washed twice and collected using Zeba™ microspin desalting columns at 1,500 g for 2 min.

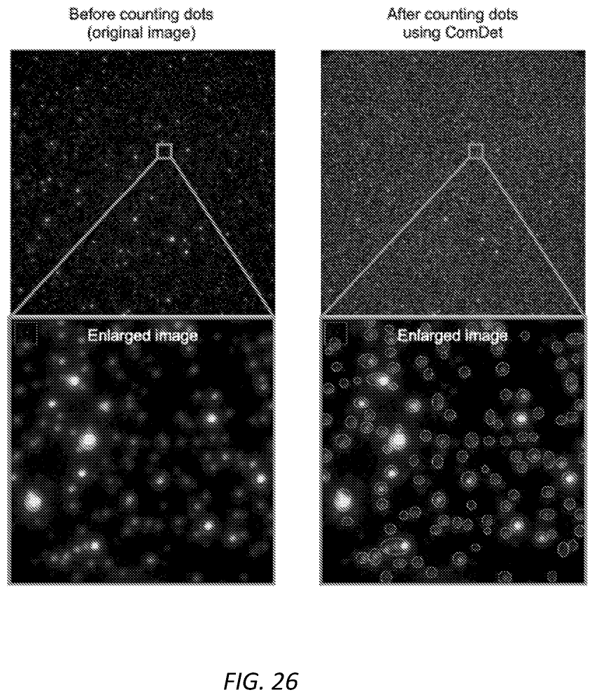

Fluorescence EV Detection: Fluorophore-labeled EV solution was dropped onto an antibody-immobilized gold substrate. After incubation for 60 min, the substrate was washed with PBS. Then, antifade mountant was dropped onto the substrate. Fluorescence images of captured EVs on gold substrates were obtained using an upright automated epifluorescence microscope (Zeiss AX10, Axio Imager.M2) equipped with a CMOS camera (C15440-20UP, Hamamatsu, Japan). The full size of a fluorescence image is about 374 μm×374 μm. For EV detection and counting, the ComDet plugin in Image J was used. In the ComDet analysis, the approximate particle size (pixel) was set to 3 and the intensity threshold (particle brightness) was set to 5. The analysis identified all fluorescence dots and output their location, size, and intensity (FIG. 26).

For the selectivity tests, OV90 and TIOSE4 (or OV90 and EVs from normal human plasma, or CaOV3 and EVs from normal human plasma) were mixed in 1× PBS (The final concentration of each EV sample was 4×10⁸ mL⁻¹.). The mixed EV solution was used for the selectivity test.

### Example 1. EV Capture on Plain Gold Surfaces

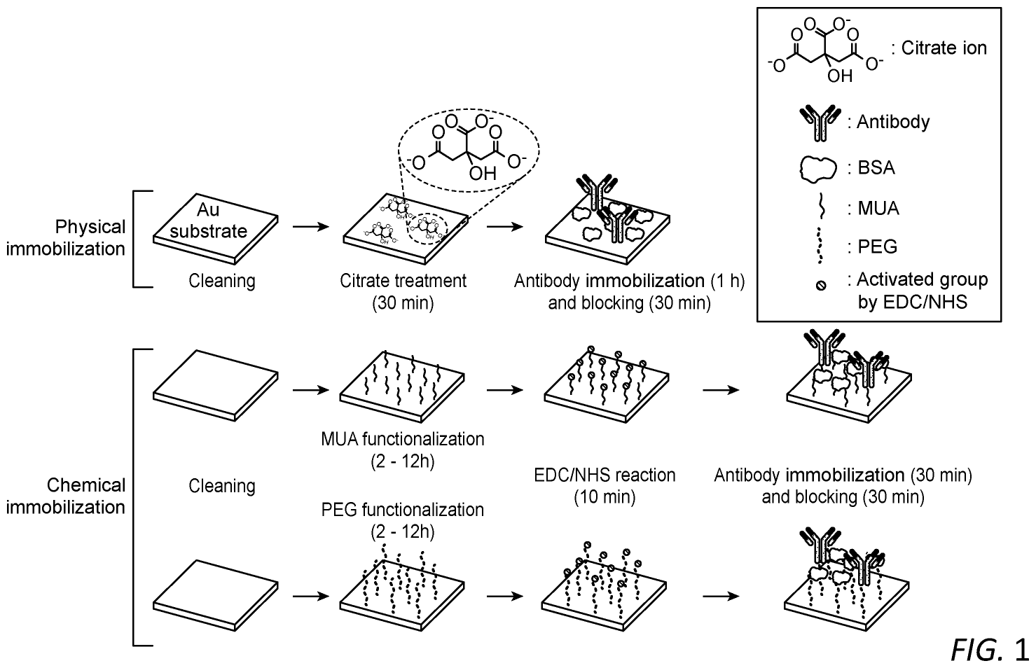

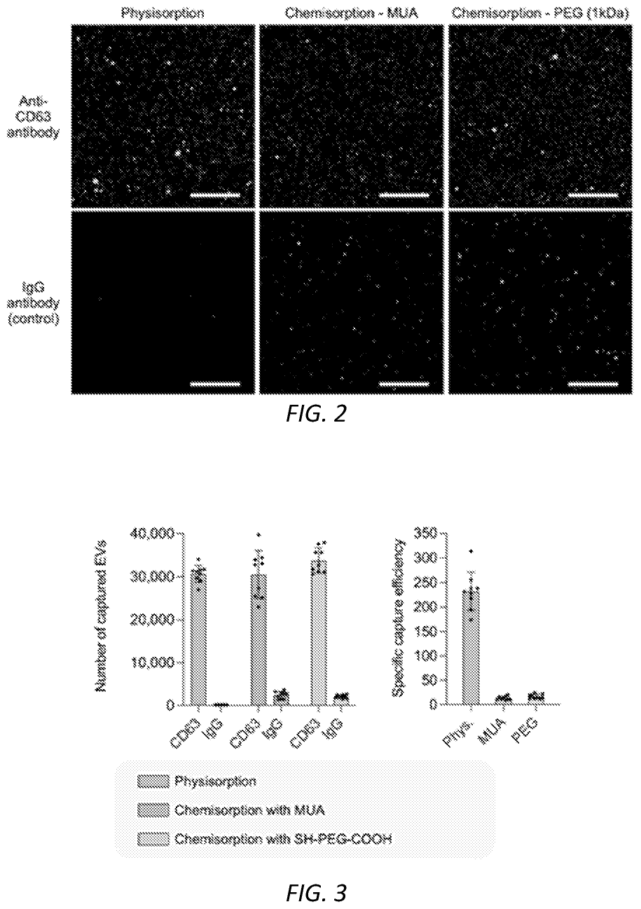

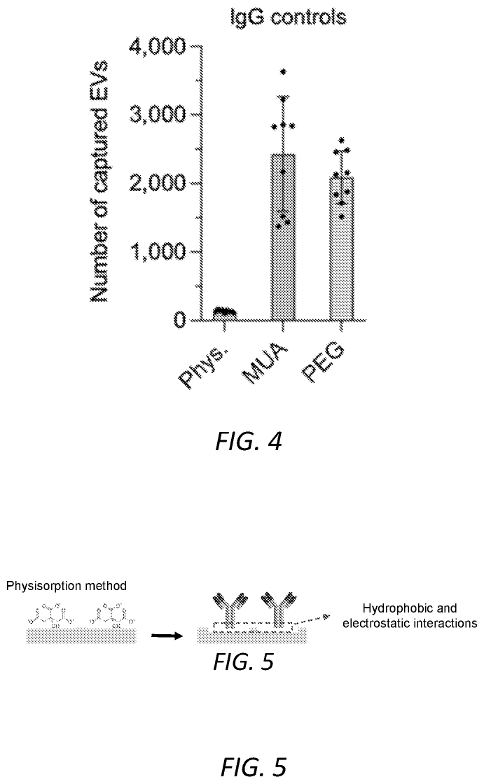

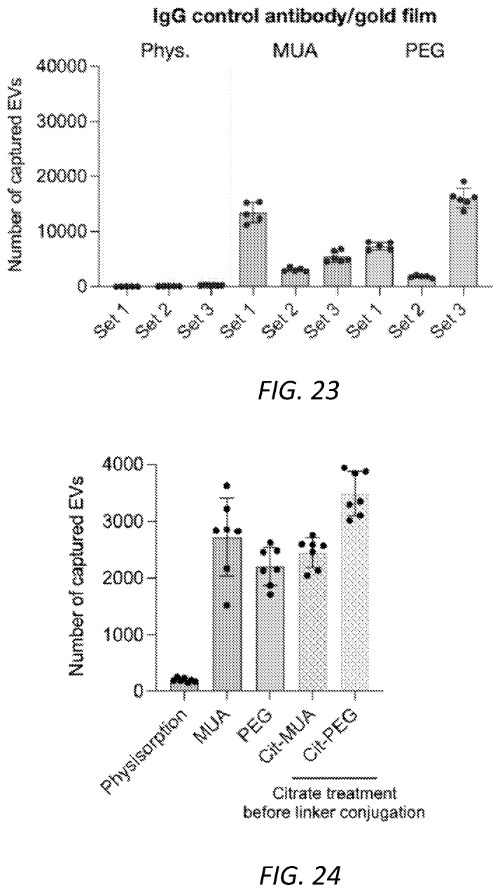

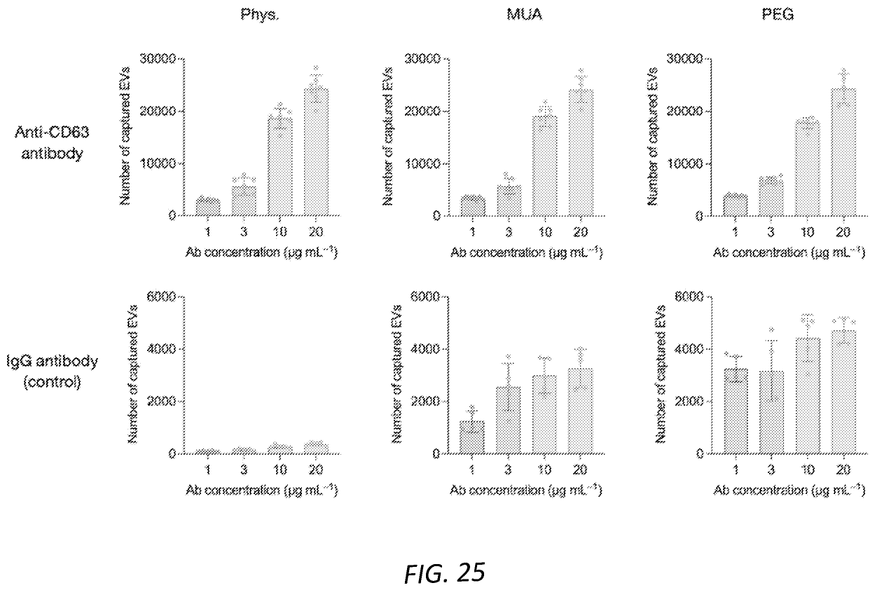

The specific and non-specific EV binding onto plain gold surfaces prepared by different methods was quantitatively evaluated. Physisorption onto a citrate-capped gold substrate (FIG. 1) immobilized antibodies via hydrophobic and electrostatic interactions between antibodies and the gold surface. This method was compared to two standard antibody immobilization methods using thiol linkers immobilized on the gold surface and subsequent antibody immobilization via EDC and sulfo-NHS. Anti-CD63 and IgG isotype control antibodies were used to measure specific and non-specific bindings of EVs, respectively. For better visualization and quantification, EVs were isolated from OV90, a human ovarian carcinoma cell line, and labeled EVs with AZDye-555-tetrafluorophenyl (TFP) esters. After incubating EVs on the antibody-immobilized gold surfaces for 60 min and subsequent washing, captured EVs were imaged and counted using a fluorescence microscope (FIG. 2). On the anti-CD63 antibody-coated surface, there were no significant differences in captured EV counts among the three methods (P=0.17, Kruskal-Wallis test; FIG. 3, left). But notably, the gold surface with physisorbed IgG antibodies had a one-order lower number of non-specifically bound EVs (mean EV count=134.9, n=9) compared to other chemisorbed IgG antibodies on gold substrates (mean EV counts=2,430 for MUA and 2,088 for PEG, n=9; FIG. 4). Moreover, the physisorption method showed better reproducibility with a coefficient of variation of 6.4% (n=9) than chemisorption methods (18.1% with MUA, 8.6% with PEG, n=9). Testing of variations in triplicate measurements showed the CV of 6.6% for the physisorption method and 7.5-20.4% for the chemisorption methods (FIGS. 22-23). Specific capture ratios (i.e., the ratio of between specifically and non-specifically captured EVs, FIG. 3, right) were calculated, and the physisorption method showed over 200 ratios, 17.1 and 13.7 times higher than MUA-and PEG-based chemisorption methods, respectively. The chemisorption methods were also tested on citrate-treated gold surfaces to determine whether the citrate treatment contributes to the lower nonspecific binding. FIG. 24 shows that the citrate treatment on the gold substrate before MUA or PEG immobilization did not significantly decrease nonspecific binding of the EVs. In addition, we tested different antibody concentrations and found that 20 μg mL⁻¹ showed the highest net EV binding (FIG. 25).

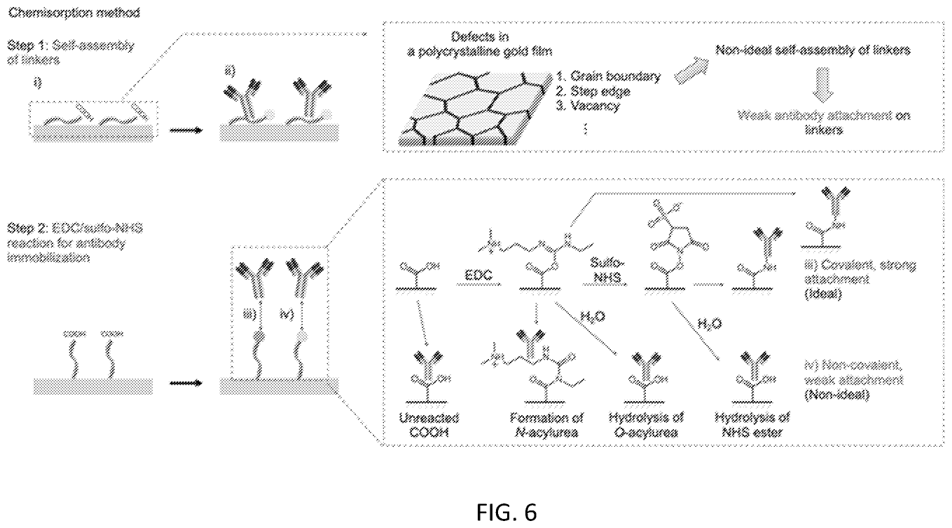

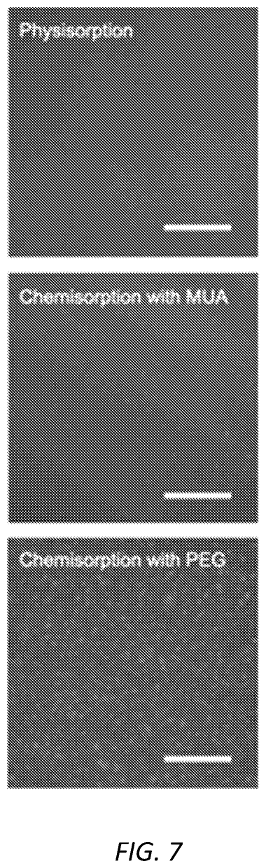

Without intending to be bound by theory, the ultra-low non-specific binding and high reproducibility could be attributed to the effective blocking of the gold surface by physisorbed antibodies and BSA (FIG. 5). The physisorption involves both hydrophobic and electrostatic interactions directly between antibodies along with blocking molecules (e.g., BSA) and gold surface (FIG. 5). As a result, uniform antibody coating was obtained, as confirmed by fluorescence imaging using AF647 dye-conjugated antibodies (FIG. 7) and good reproducibility for EV capture (FIG. 3, left). In contrast, chemisorption methods with thiol linkers have can result in weak immobilization of antibodies and blocking proteins (FIG. 6) that can be displaced by EVs as non-specific binding. Chemisorption methods typically involve two steps: self-assembly of a linker layer and covalent antibody conjugation. In this process, non-ideal linker formation and non-covalent antibody attachment can occur for different reasons: First, the self-assembly of thiol linkers on the gold surface may possess defective formation of linkers. Gold films prepared by evaporation or sputtering contain many defect sites, such as grain boundaries, step edges, and vacancy (FIG. 8). Self-assembly of thiol linkers can be defectively formed at the defect sites (FIG. 6, i), and as a result, antibodies can weakly bind to carbon (or polymer) chains of the linkers (FIG. 6, ii) instead of covalent immobilization. Second, an EDC/NHS reaction is fast and unstable, which can result in the formation of not only amine-reactive groups (O-acylurea, FIG. 6, iii) but also non-amine-reactive groups (FIG. 6, iv). Specifically, rapid hydrolysis of reactive groups can occur during EDC/NHS activation in water, leading to forming carboxyl groups and urea derivatives such as N-acylurea. Antibodies cannot be covalently attached on such non-amine-reactive groups, and EVs can replace the weakly bound antibodies and blocking materials. Fluorescence imaging showed non-uniform immobilization of AF647-conjugated antibodies on gold films when chemisorption methods were used possibly due to the defective formation of linkers and the byproduct production, while the physisorption method produced a uniform antibody coverage on the same gold surface (FIG. 7).

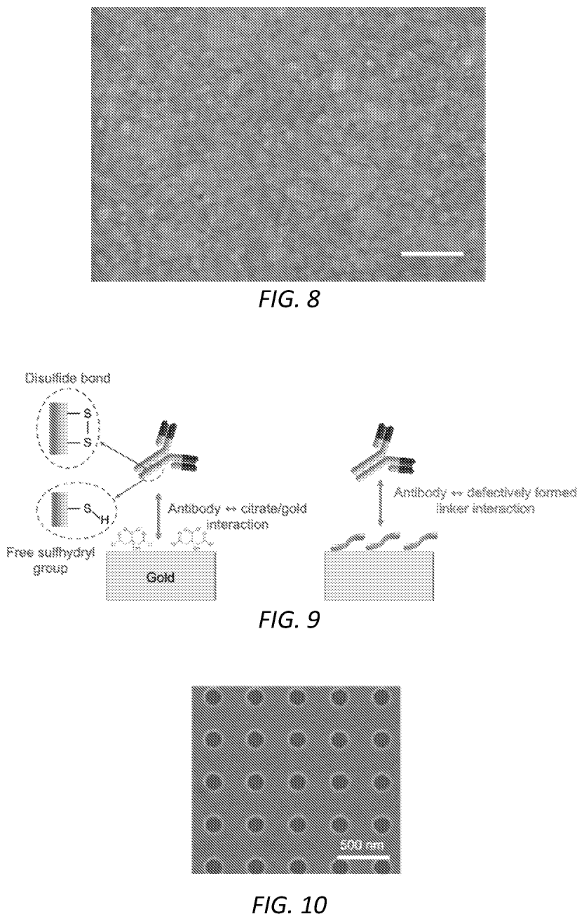

Notably, antibodies and BSA contain cysteine residues (e.g., including free sulfhydryl group) that have a high affinity to gold. Without intending to be bound by theory, in the case of direct antibody immobilization, in addition to hydrophobic and electrostatic interactions, cysteine residues can contribute to irreversible antibody immobilization on the gold surface (FIG. 9). These residues could contribute to the uniform and stable antibody immobilization achieved using the physisorption method, which led to much lower non-specific EV bindings.

### Example 2. EV Detection on Nano-Patterned Gold Substrates

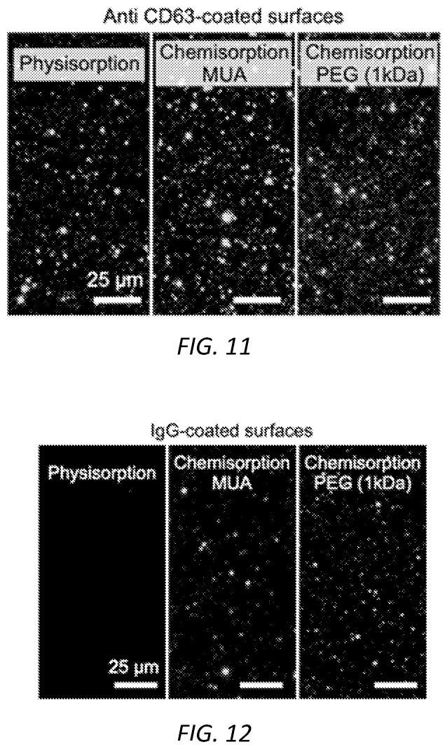

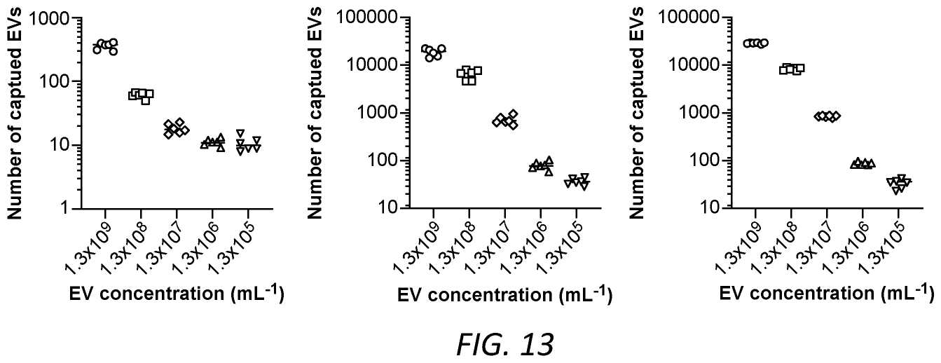

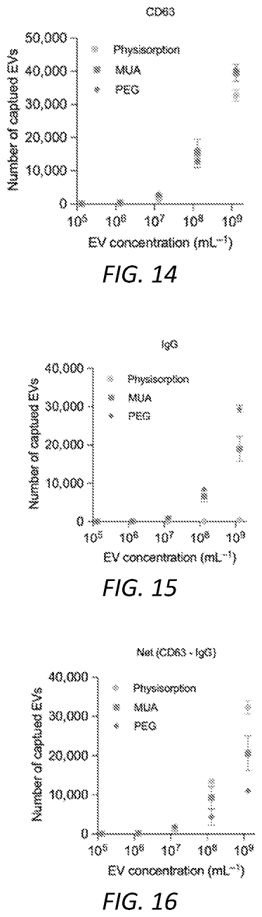

The three methods of Example 1 were next tested on nano-patterned gold substrates for EV detection. Gold nanowell structures with 200-nm hole size and 500-nm periodicity (FIG. 10) were used. The nanoplasmonic structures have previously shown excellent EV detection sensitivity. Direct physisorption and chemisorption with MUA or SH-PEG-COOH were applied to immobilize anti-CD63 and IgG control antibodies, and then EV binding on the surfaces was measured. In titrating EV concentrations, while the number of EVs captured on anti-CD63 antibody-coated gold nanowell substrates showed similarly regardless of antibody immobilization methods (FIG. 11), the direct physisorption method showed significantly lower amounts of non-specific EV binding on IgG-coated gold nanowell substrates compared to those prepared by chemisorption methods (FIGS. 12 and 13). At a high EV concentration (˜109 EV mL-1), the direct immobilization method showed a >50-fold lower non-specific EV binding (363±43, n=6) compared to the other substrates prepared by chemisorption methods (18937±3289, n=6 with MUA; 29439±957, n=6; P<0.0001, one-way ANOVA; FIGS. 14 and 15). With the lower non-specific binding, the direct immobilization method showed significantly higher specific EV binding, calculated by subtracting EV counts on IgG from the counts on CD63 antibody-coated surfaces, than chemisorption methods (P<0.0001, one-way ANOVA; FIG. 16). In comparison between plain and nanowell structures, all three methods showed slightly higher non-specific binding on nanowell structures (a 2-fold increase with the direct physisorption method and a 10-fold increase with the chemical methods). Without intending to be bound by theory, this could be attributed to the curvatures around nanowell structures where the more defective formation of self-assembled linkers can occur, leading to more non-specific EV bindings. Therefore, the physisorption method could be advantageous over chemisorption methods for antibody immobilization on gold nanostructures.

### Example 3. EV Capture Specificity on Nano-Well Substrates

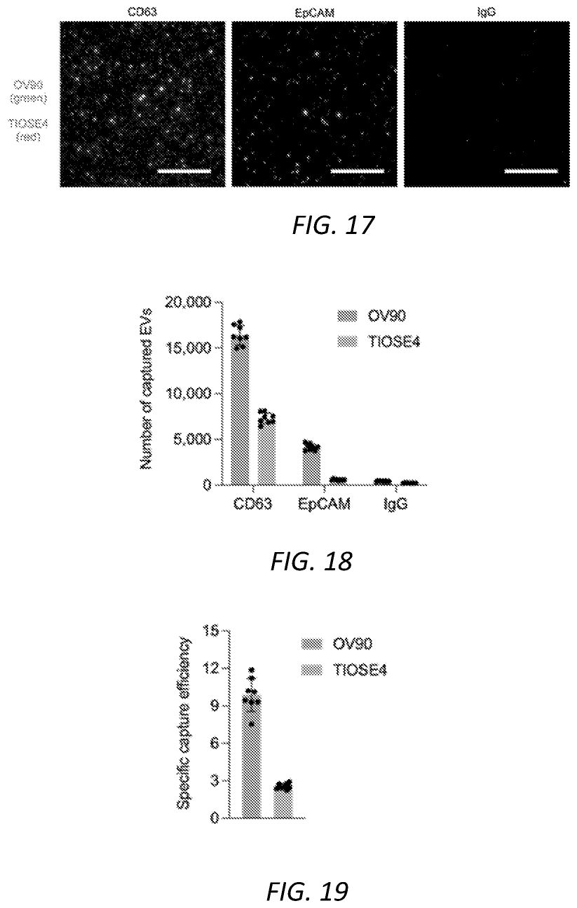

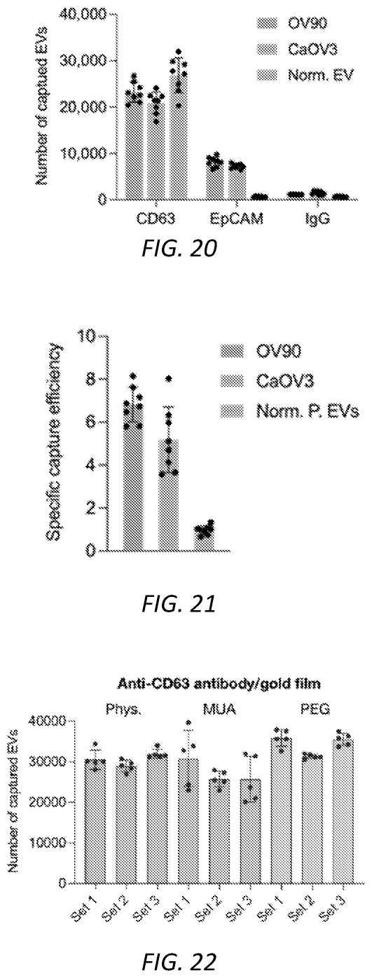

Next, the specificity to capture and detect cancer-derived EVs on the nanowell immunogold substrate was tested. EpCAM is highly expressed in EVs from ovarian cancer patients differentiated and thus could be used as a diagnostic biomarker. Whether nanowell plasmonic substrates prepared by the proposed physisorption method could specifically detect cancer-derived EVs in high backgrounds of EVs from benign cells or normal plasma was tested. EVs from ovarian cancer (OV90) and benign (TIOSE4) cell lines were mixed in a 1:1 ratio and applied on nanowell substrates coated with anti-CD63, anti-EpCAM, and IgG control antibodies by the physisorption method. For quantification, OV90 EVs were labeled with AZDye 555 and TIOSE4 EVs were labeled with AZDye 647 (FIG. 17). Both OV90 and TIOSE4 EVs were captured on the anti-CD63 coated surfaces, as CD63 is a universal marker for EVs (FIG. 18). However, on the substrates coated with anti-EpCAM antibody for cancer-derived EV detection, a significantly higher number of OV90 EVs were captured (P=0.0002, Mann-Whitney t-test), while the captured TIOSE4 EVs counts were comparable with those on the IgG control antibody-coated substrates (FIG. 19). The result demonstrates specific capture and detection of cancer-derived EVs with little non-specific binding on the gold nanowell substrates; this could be used to differentiate ovarian cancer from benign cases. The assay was also tested for OV90 and CaOV3, ovarian cancer-derived EVs, mixed with host cell-derived EVs from a healthy normal plasma sample (FIGS. 20 and 21). The results showed that cancer-derived EVs can be effectively captured on the nanowell substrates coated with anti-EpCAM antibodies with little non-specific binding of host cell-derived EVs below the counts on the IgG control antibody-coated surface. For substrates coated with anti-CD63 antibodies, both cancer cell-and host cell-derived EVs were captured.

### Example 4. Soluble Protein Detection

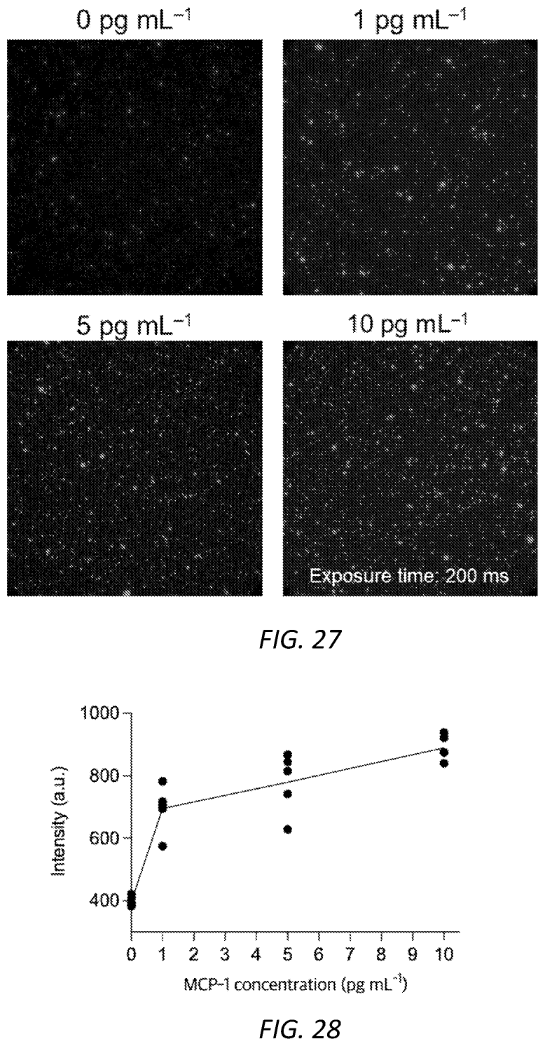

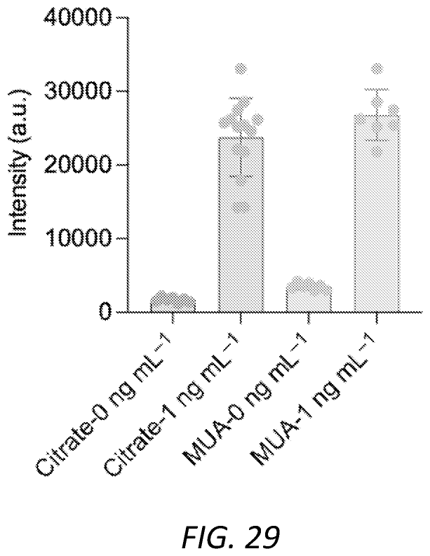

The direct physisorption (citrate-based) and chemisorption (MUA-based) methods of Example 1 were tested on nano-patterned gold substrates for detection of a soluble protein (monocyte chemoattractant protein-1, “MCP-1”). Direct physisorption or chemisorption with MUA was applied to immobilize anti-MCP-1 antibodies to form sensors, and then protein binding on the sensor surface was measured by chemifluorescence sandwich assay for solutions of 0 pg/mL, 1 pg/mL, 5 pg/mL, 10 pg/mL, and 1 ng/mL MCP-1 with fluorophore-conjugated anti-MCP-1 detection antibodies. Comparison of fluorescence after contacting solutions of 0 pg/mL, 1 pg/mL, 5 pg/mL, and 10 pg/mL with substrates prepared by direct physisorption of anti-MCP-1 antibodies (FIGS. 27 and 28) show low non-specific binding. As shown in FIG. 29, direct physisorption (citrate-based surface chemistry) resulted in better signal-to-noise ratios for protein detection, as compared to the MUA-based chemisorption method.

### Example 5. EV Detection on Nanoparticle-on-Nanopillar (NPOP) Substrate

The direct physisorption (citrate-based) and chemisorption (SH-PEG-COOH-based) methods of Example 1 were tested on an NPOP (Au nanoparticles on Au nanopillars) plasmonic substrate for detection of EVs from the SkBr3 breast cancer cell line (isolated by size exclusion chromatography) spiked in healthy plasma. Direct physisorption or chemisorption was applied to immobilize QUAD capture antibodies (antibody mixture against EpCAM, EGFR, HER2, and MUC1) to form sensors.

Control samples were prepared by contacting the sensors with PBS buffer free from EVs, or with healthy plasma lacking the tumor-derived EVs. Test samples were prepared by contacting the substrate with healthy plasma spiked with SkBr3 EVs, or SkBr3 EVs pre-labeled with TFP-AF555 for fluorescence detection. Captured EVs were immunolabeled for HER2 and CD-mix (CD63, CD9, CD81), and their colocalized signals were analyzed.

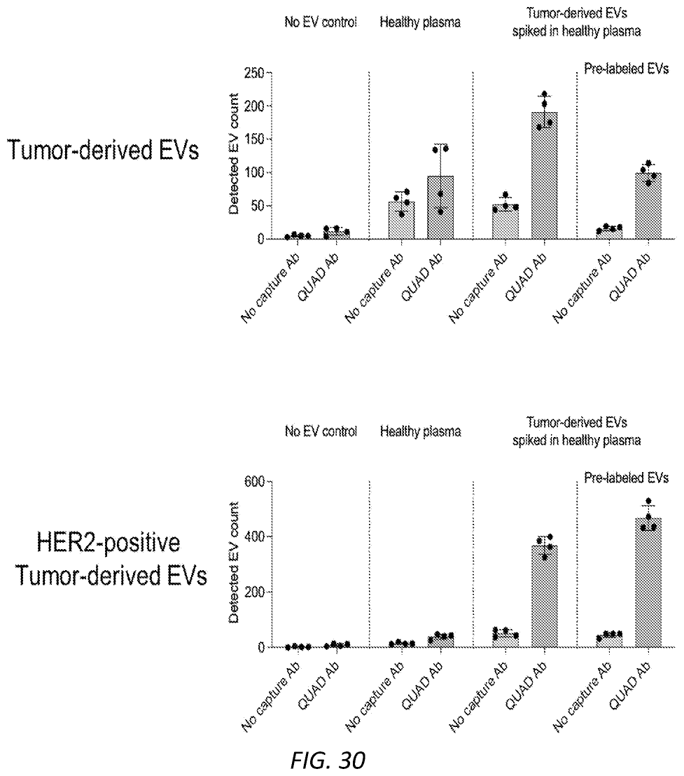

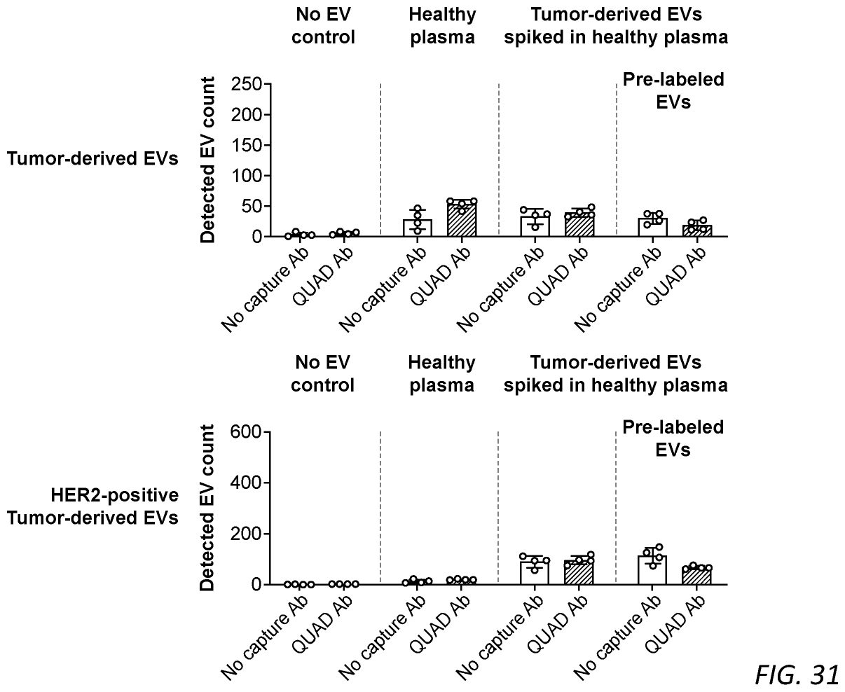

Results for the directly physisorbed sensors and the chemisorbed sensors are shown in FIGS. 30 and 31, respectively. The results show that the assay using the directly physisorbed substrate shows good sensitivity and specificity for detecting tumor-derived EVs and HER2 or CD-positivity within the captured tumor-derived EVs, as compared to substrates formed using chemisorption (PEG-based surface chemistry).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing a sensor, the method comprising
contacting an antibody with a surface of a film comprising an ionic compound and a metal selected from gold, silver, platinum, copper, and any combination thereof; and then
contacting a blocking agent with the surface of the film to form the sensor.

2. The method of claim 1, wherein the metal is silver or gold.

3. The method of claim 1, wherein the metal is gold.

4. The method of claim 1, wherein the ionic compound comprises an ammonium group, a carboxylate group, or an azide group.

5. The method of claim 1, wherein the ionic compound comprises a carboxylate group.

6. The method of claim 5, wherein the ionic compound comprises a citrate ion, an acetate ion, a succinate ion, a glutarate ion, or any combination thereof.

7. The method of claim 5, wherein the ionic compound comprises a citrate ion.

8. The method of claim 1, wherein the surface consists essentially of the metal and the ionic compound.

9. The method of claim 1, further comprising, before contacting the antibody, forming the surface by contacting a precursor film comprising the metal with the ionic compound.

10. The method of claim 9, comprising contacting the precursor film with an aqueous solution of the ionic compound.

11. The method of claim 1, comprising
contacting the surface of the film with an aqueous solution of the antibody; and then
contacting the surface of the film with an aqueous solution of the blocking agent.

12. The method of claim 1, wherein the surface is flat.

13. The method of claim 1, wherein the surface comprises nano-scale features.

14. A method of physisorbing an antibody onto a film, the method comprising contacting the antibody with a surface of the film consisting essentially of an ionic compound and a metal selected from gold, silver, platinum, copper, and any combination thereof.

15. The method of claim 14, wherein the ionic compound comprises an ammonium group, a carboxylate group, an azide group, or any combination thereof.

16. The method of claim 15, wherein the ionic compound comprises a carboxylate group.

17. The method of claim 16, wherein the ionic compound comprises a citrate group.

18. The method of claim 14, wherein the metal is gold or silver.

19. The method of claim 14, wherein the metal is gold.

20. The method of claim 14, wherein the surface is flat.

21. The method of claim 14, wherein the surface comprises nano-scale features.

22. A sensor, prepared according to the method of claim 1.

23. A film and an antibody physisorbed onto the surface thereof, prepared according to the method of claim 14.

24. A sensor, comprising:
a film having a sensing surface;
an antibody physisorbed on the sensing surface; and
a blocking agent physisorbed on the sensing surface;
wherein the film comprises a metal selected from gold, silver, platinum, copper, and any combination thereof, and the metal makes up at least a portion of the sensing surface.

25. The sensor of claim 24, wherein the metal is gold or silver.

26. The sensor of claim 24, wherein the metal is gold.

27. The sensor of claim 24, wherein the sensing surface comprises an ionic compound.

28. The sensor of claim 27 wherein the ionic compound comprises an ammonium group, a carboxylate group, or an azide group.

29. The sensor of claim 27, wherein the ionic compound comprises a carboxylate group.

30. The sensor of claim 29, wherein the ionic compound comprises a citrate ion, an acetate ion, a succinate ion, a glutarate ion, or any combination thereof.

31. The sensor of claim 29, wherein the ionic compound comprises a citrate ion.

32. The sensor of claim 27, wherein the sensing surface consists essentially of the metal and the ionic compound.

33. The sensor of claim 24, wherein the antibody has a dissociation constant of 0.1 nM to 500 nM for the sensing surface.

34. The sensor of claim 24, wherein the antibody has a dissociation constant of 0.1 nM to 200 nM for the sensing surface.

35. The sensor of claim 24, wherein the antibody is capable of binding to a biomarker for cancer, neurodegenerative disease, blood/vascular disease, infection, inflammation, or wound healing.

36. The sensor of claim 24, wherein the antibody is capable of binding to a biomarker for cancer.

37. The sensor of claim 24, wherein the antibody is capable of binding to EpCAM, EGFR, MUC-1, HER2, CD24, CA125, CD45, CD63, CD81, CD9, or DAPI.

38. The sensor of claim 35, wherein the iomarker comprises an extracellular vesicle.

39. The sensor of claim 35, wherein the biomarker comprises a soluble protein.

40. The sensor of claim 39, wherein the soluble protein comprises a cytokine.

41. The sensor of claim 24, wherein the blocking agent has a dissociation constant of 0.1 nM to 500 nM for the sensing surface.

42. The sensor of claim 24, wherein the blocking agent has a dissociation constant of 0.1 nM to 200 nM for the sensing surface.

43. The sensor of claim 24, wherein the blocking agent comprises bovine serum albumin, fetal bovine serum, goat serum, steelhead salmon serum, non-fat milk, SuperBlock™ (Thermo Scientific), AdvanBlock™ (Advantsta Inc.), or any combination thereof.

44. The sensor of claim 24, wherein the blocking agent comprises bovine serum albumin.

45. The sensor of claim 24, wherein
the sensing surface comprises binding-accessible domains unoccupied by the antibody or the blocking agent; and
the binding-accessible domains have an average size of 1 nm to 50 nm.

46. The sensor of claim 24, wherein the sensing surface is flat.

47. The sensor of claim 24, wherein the sensing surface comprises nano-scale features.

48. The sensor of claim 47, wherein the nano-scale features comprise holes, ridges, channels, wells, pillars, pyramids, cones, particles, or any combination thereof.

49. The sensor of claim 47, wherein the nano-scale features comprise wells.

50. The sensor of claim 49, wherein the wells have an average diameter of 50 nm to 1000 nm, and an average depth of 50 nm to 1000 nm.

51. The sensor of claim 47, wherein the nano-scale features comprise pillars.

52. The sensor of claim 51, wherein the pillars have an average diameter of 20 nm to 1000 nm, and an average height of 20 nm to 1000 nm.

53. The sensor of claim 47, wherein the nano-scale features comprise particles.

54. The sensor of claim 53, wherein the particles have an average diameter of 20 nm to 500 nm.

55. The sensor of claim 45, wherein adjacent nano-scale features are spaced apart by an average distance of 50 nm to 1 μm.

56. The sensor of claim 55, wherein the film has an average thickness of 10 nm to 1 μm.

57. The sensor of claim 24, further comprising a substrate, wherein the film is disposed on the substrate and the sensing surface faces away from the substrate.

58. The sensor of claim 57, wherein the substrate comprises silicon.

59. The sensor of claim 58, wherein the substrate comprises crystalline silicon or crystalline silicon nitride.

60. The sensor of claim 58, wherein the substrate comprises an adhesion layer, the film is disposed on the adhesion layer, and the sensing surface faces away from the adhesion layer.

61. The sensor of claim 60, wherein the adhesion layer has an average thickness of 1 nm to 50 nm.

62. The sensor of claim 60, wherein the adhesion layer comprises titanium.

63. The sensor of claim 57, wherein the substrate comprises glass or plastic.
